(19) United States
(12) Patent Application Publication
(10) Pub. No.: US 2019/0025011 A1
(43) Pub. Date: Jan. 24, 2019

(54) SAFETY DEVICE FOR NAILS AND ASSOCIATED METHOD

(71) Applicant: Mauro Antonio PEDROLI, Lugano (CH)

(72) Inventor: Mauro Antonio PEDROLI, Lugano (CH)

(73) Assignee: Mauro Antonio PEDROLI, Lugano (CH)

(21) Appl. No.: 16/071,592

(22) PCT Filed: Jan. 18, 2017
(86) PCT No.: PCT/IB2017/050259

§ 371 Date: Jul. 20, 2018

(30) Foreign Application Priority Data
Jan. 22, 2016 (CH) 00089/16

(51) Int. Cl. F41B 15/00; A45D 31/00; A61B 10/02

(52) U.S. Cl. CPC F41B 15/00; A45D 2200/00; A61B 10/0266; A45D 31/00

(57) ABSTRACT

Safety device (100) per nails (101), said device (100) being characterized in that it comprises a sheet (103) in resistant material, developing principally along a first and a second axis (X, Y), suitable for being installed over of a nail of a person and below of a fake nail (102), said sheet (103) in resistant material comprising in an its first end cutting and/or pointed means (104; 104a, 104b, 104c) susceptible of realizing a receptacle where containing portions of skin of an aggressor.

### FIELD OF THE INVENTION

The present invention generally refers to safety devices for people. In detail, the present invention concerns a safety device for nails.

The present invention further concerns a fake nail that integrates said safety device.

The present invention finally concerns the method of application of said device to the nails of a person.

### BACKGROUND ART

It is known that people, often but not only female, are subject to aggressions. In particular, is further known that when aggressed people typically defend themselves from the aggression with naked hands. During said defense, portions of skin of the aggressor insinuate below the nails, provided that these last have sufficient length.

From said portions of skin it is possible extract portions of DNA useful for retrieving or anyway classify the aggressor in case he is not immediately captured by law enforcement agencies.

Sometimes, the hands and in particular the nails of the offended person therefore become an instrument of defense, in particular the first—in particular for unarmed people.

The applicant has observed that, often, sometimes short nails do not offer a sufficient area for keeping portions of skin of the aggressor useful to retrieve DNA thereof or anyway to offend him sufficiently.

The scope of the present invention is therefore to describe a safety device, suitable to be applied to nails of a person, that allows for solving the aforementioned drawbacks.

A further scope of the present invention is furthermore to describe a fake nail, suitable for being used together with said device on a true nail of the person, in such a way to solve the aforementioned drawback.

A further scope of the present invention is finally to describe a method of application of said device that allows for solving the aforementioned drawbacks.

### SUMMARY OF THE INVENTION

To the end of increase primarily the safety of people that in case offended can defend themselves with the hands keeping portions of skin below the nails so that to guarantee sufficient material for the DNA analysis, according to the present invention is described a safety device for nails, said device being characterized in that it comprises a sheet in resistant material, developing principally along a first and a second axis, suitable for being installed over of a nail of a person and below of a fake nail, said sheet in resistant material comprising in an its first end cutting means and/or pointed susceptible of realizing a receptacle where containing portions of skin of an aggressor.

According to a first aspect of the invention, said sheet in resistant material is realized in metallic material.

According to a further aspect of the invention, said sheet in resistant material is substantially planar.

According to a further aspect of the invention, said sheet has a curvature and/or can be bent respective to said plane with a radius compatible with a radius of curvature of the said nail.

According to a further aspect of the present invention, said cutting means and/or pointed comprise a pointed pin or a hook.

In a preferred embodiment of the present invention, said pointed pin or hook extends substantially orthogonally respective to said plane.

According to the present invention is furthermore realized a fake nail, suitable for being applied over a nail of a person; said fake nail having a first upper face and a second lower face counterposed to said first upper face, wherein on said second face is applied a safety device according to any of the features previously described.

In detail, according to an preferred aspect of the present invention, said second face has a recess suitable for housing at least part of the thickness of said safety device.

According to the present invention is furthermore realized a method of application of a safety device for nails, said method comprising a step of gluing of a safety device according to any of the features described above over a nail of a person, and a step of superimposition of a fake nail over said safety device, so that this last rests in a sandwich defined at the bottom by the true nail of the person and at the top by the fake nail

According to an aspect of the present invention, said method comprises a step of bending said safety device so that to make it assume a radius of curvature corresponding or substantially corresponding to the radius of the said nail.

More in detail, said bending takes place on a smaller axis of said safety device. In a further aspect of the present invention, said method comprises a step of positioning of said safety device within a recess arranged in a lower concave wall of said fake nail, said lower concave wall being counterposed to a upper convex wall of the said fake nail.

In a further step of the present invention, said method comprises a step of orienting of cutting and/or pointed means susceptible of realizing a receptacle for containing portions of skin of an aggressor in a direction which is orthogonal to the direction or plane of maximum extension of said device.

### DESCRIPTION OF THE FIGURES

Further features of the invention will be described in one or more preferred and non-limiting embodiments referring to the annexed figures wherein:

FIG. 1 shows a plan view of a terminal portion of a finger of a person, with a nail, a fake nail and a safety device object of the present invention;

FIG. 2 shows a lateral view of the device object of the present invention in an its preferred and non-limiting embodiment;

FIG. 3 shows a lateral view of the device object of the present invention in an its perspective view object of the present invention;

FIG. 4 shows a front view of a finger of a person provided with of the device object of the present invention in an its preferred and non-limiting embodiment.

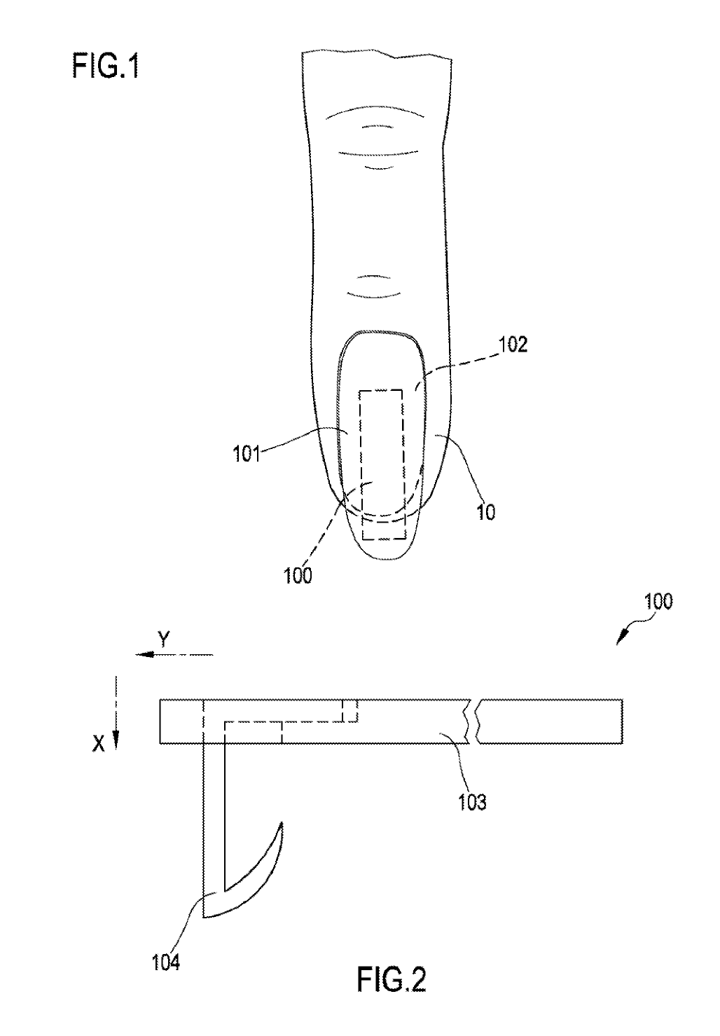

### DETAILED DESCRIPTION OF THE INVENTION

Referring to the annexed figures, with the reference number 100 is shown in its complex a safety device 100 for nails 101.

The safety device 100 comprises a sheet 103 that in a first preferred and non-limiting embodiment develops along a plane defined by a first axis X and a second axis Y orthogonal one another.

The sheet 103 is realized in resistant material, preferably but in a non-limiting extent in harmonic steel or in a synthetic material, for example aramid, offering a resistance sufficient to not to tear or damage with a force equal to that can be exerted with the fingers of a hand of an adult person.

The safety device 100 is suitable in detail to be installed over the true nail 101 of a person and below the fake nail 102 that is superimposed thereto, forming a sandwich.

Advantageously, the safety device 100 object of the present invention has cutting and/or pointed means that allow to realize on one hand a receptacle where containing or retaining portions of skin of an aggressor and, at the same time, to cause a slight wounding, beyond to the wounding that could be made with the sole action of a human nail.

In detail, said cutting and/or pointed means can comprise pins and/or hooks, that preferably extend principally along a direction orthogonal respective to the plane on which said sheet substantially lies.

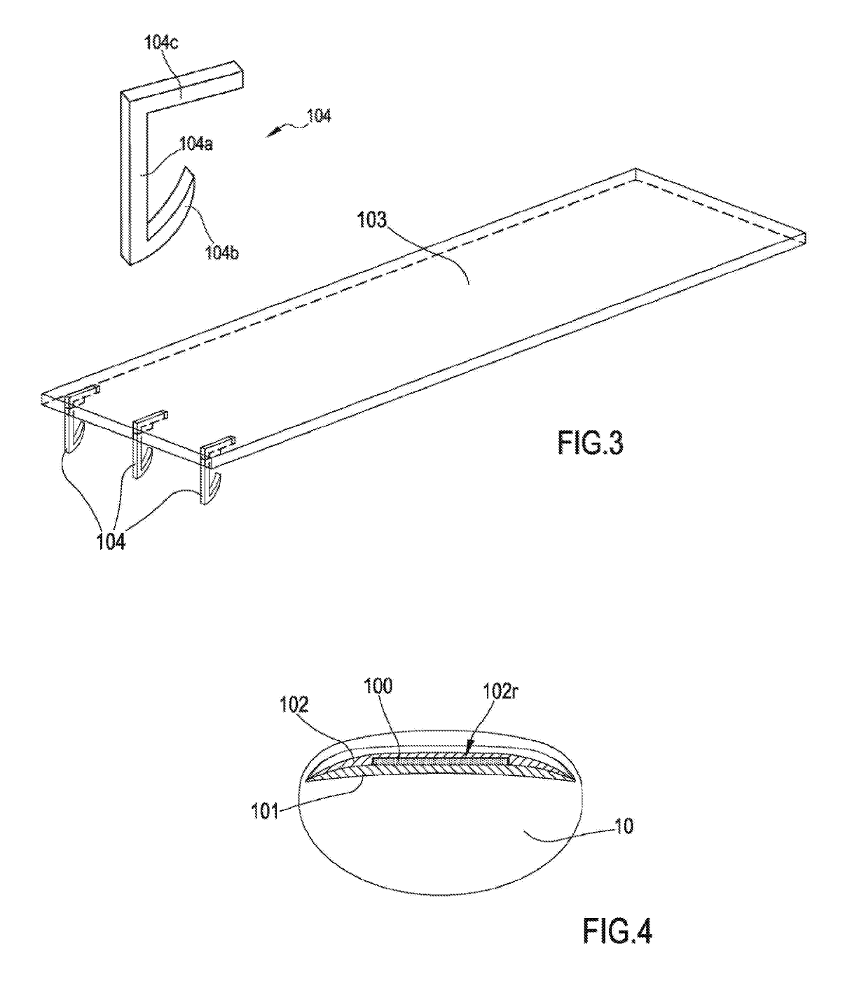

In a preferred and non-limiting embodiment of the present invention, the hooks 104 that are depicted in FIG. 3 are positioned in a position of substantial front end portion of the said sheet 103, and comprise a first portion 104c, a second portion 104a and a third portion 104b. The first portion 104c is the one that is fixed in correspondence or within the sheet 103 itself.

The second portion 104a extends orthogonally to the first portion 104c, and the third portion 104b is directed backwards and forms the veritable hook within which retaining the portions of skin.

Even if in FIG. 3 is shown an embodiment of the safety device 100 object of the present invention that integrates 3 hooks, said number shall not be intended for any reason in a limiting extent.

In an embodiment alternative to that which is described above, the sheet 103, even if extending substantially along a plane, is bent and forms—when observed frontally along the direction of smaller extension respective to the axis of maximum extension, a bending substantially equal to the one which is defined by the nail of the person.

This advantageously allows to obtain a greater easiness of installation of the object of the present invention 100 on the true nail 101 of the person, in particular because it follows the shape thereof In case the material of the sheet 103 can be deformed, this can be adapted to bring the shape and the radius of curvature of the nail 101 of the person.

The thickness of the sheet 103 preferably does not exceed 1 mm, this allowing a greater aesthetic effectiveness.

Furthermore, the vertical extension of the second portions 104a of the hook does not exceed the 2 or 3 mm.

According to the present invention is furthermore realized also a fake nail 102 to be applied on a true nail 101 of a hand of a person. The fake nail 102 has a first face convex upper and a second face concave lower than, opposite respective to the first face convex upper.

In detail, on said second lower concave face is defined a recess 102r with a shape substantially equal to that of the sheet 103. Said recess 102 is therefore suitable in use to receive and house the sheet 103 for at least part of its thickness. This advantageously allows for reducing the thickness that creates summing that of the sheet 103 to that of the fake nail 102 in case there is not the recess 102r. Preferably, furthermore, the recess 102r extends along at least part of the direction of maximum extension of the fake nail.

In a preferred and non-limiting embodiment of the present invention, the installation of the safety device 100 object of the present invention follows a proceeding as follows.

At first the true nail 101 is prepared with a proceeding suitable to clean it and in case roughen a bit its upper portion so that to increase the grip of the sheet 103. Subsequently to the true nail 101, the sheet 103 is superimposed, at first in case yet arranged within the recess 102 of the fake nail 102, which is therefore arranged over the two preceding elements.

It is therefore realized a sandwich formed by the true nail 101 in the bottom part, by the sheet 103 with hooks 104 and by the fake nail 102 in the top part.

If necessary, before superimposing the fake nail 102, the sheet 103 is bent for allowing it to assume the radius of curvature of the true nail 101.

Furthermore, to the end of ensuring a greater grasping, hooks 104 are oriented towards the true nail 101, i.e. the portion 104a is made point downwards and not upwards where there is the fake nail 102.

The advantages of the device object of the present invention are clear in the light of the description that precedes.

It advantageously allows of increasing the safety and the defensive capacity of people, and in particular women, in case they are subjected to an aggression, in particular when unarmed.

Therefore, not only the offence capacity of the traditional nail is increased, but also there is favouring in using a nail for creating an environment suitable for collecting fragments of skin of the aggressor that allow for the authorities of police an easier identification thereof, in case this is not possible or anyway difficult by the aggressed user.

It is finally clear that to the object of the present invention can be applied additions, modifications or variants, obvious for a skilled person, without for this departing from the scope of protection provided by the annexed claims.

1. Safety device (100) per nails (101), said device (100) being characterized in that it comprises a sheet (103) in resistant material, developing principally along a first and a second axis (X, Y), suitable for being installed over of a nail of a person and below of a fake nail (102), said sheet (103) in resistant material comprising in an its first end cutting and/or pointed means (104; 104a, 104b, 104c) susceptible of realizing a receptacle where containing portions of skin of an aggressor.

2. Safety device (100) for nails (101) according to claim 1, characterized in that said sheet in resistant material is realized in metallic material.

3. Safety device (100) for nails (101) according to claim 1, characterized in that said sheet (103) in resistant material is substantially planar.

4. Safety device (100) per nails (101) according to any of the preceding claims, characterized in that said sheet (103) has a curvature and/or can be bent respective to said plane (X, Y) with a radius compatible with a radius of curvature of the said nail.

5. Safety device (100) per nails (101) according to any of the preceding claims, characterized in that said cutting means and/or pointed (104; 104a, 104b, 104c) comprise a pointed pin or a hook (104).

6. Safety device (100) for nails (101) according to claim 5, characterized in that said pointed pin or hook (104) extends substantially orthogonally respective to said plane.

7. A fake nail (102), suitable for being applied over a nail (101) of a person; said fake nail (102) having a first upper face and a second lower face that is counterposed to said first upper face, wherein on said second face is applied a safety device (100) according to any of the preceding claims.

8. Fake nail (102) according to claim 7, characterized in that said second face has a recess (102r) suitable to house at least part of the thickness of said safety device (100).

9. Method of application of a safety device (100) per nails (101), said method comprising a step of gluing or fixing a safety device (100) according to any of the preceding claims 1-6 over a nail (101) of a person, and a step of superimposition of a fake nail (102) al si over of said safety device, so that this last rests in a sandwich defined at the bottom by the true nail (101) of the person and at the top by the fake nail (102).

10. Method of application of a safety device (100) for nails (101) according to claim 9, characterized in that it comprises a step of bending of said safety device so that to make it assume a radius of curvature corresponding or substantially corresponding to the radius of said nail (101).

11. Method of application of a safety device (100) for nails (101) according to claim 10, characterized in that said bending takes place on a smaller axis of said safety device.

12. Method of application of a safety device (100) for nails (101) according to claim 10, characterized in that it comprises a step of positioning of said safety device (100) within a recess arranged in a lower concave wall of said fake nail (102), said lower concave wall counterposing to an upper convex wall of said fake nail (102).

13. Method of application of a safety device (100) for nails (101) according to claim 10, characterized in that it comprises a step of orienting cutting and/or pointed means susceptible of realizing a receptacle where containing portions of skin of an aggressor in a direction orthogonal to the direction or plane of maximum extension of said safety device (100).
